(19) United States
(12) Patent Application Publication
(10) Pub. No.: US 2007/0218079 A1
(43) Pub. Date: Sep. 20, 2007

(54) METHOD TO INDUCE RNAI IN PROKARYOTIC ORGANISMS

(75) Inventor: Volker Patzel, HANAU (DE)

Correspondence Address: ROTHWELL, FIGG, ERNST & MANBECK, P.C., 1425 K STREET, N.W., SUITE 800, WASHINGTON, DC, 20005, US

(73) Assignee: MAX-PLANCK-GESELLSCHAFT ZUR FOERDERUNG DER WISSENSCHAFTEN E.V., MUENCHEN (DE)

(21) Appl. No.: 11/596,176

(22) PCT Filed: May 12, 2005
(86) PCT No.: PCT/EP05/05188

§ 371 Date: Nov. 13, 2006

(30) Foreign Application Priority Data
May 12, 2004 (EP) 04 011 319.3

(51) Int. Cl. A01K 67/027; A61K 39/02; C12N 1/21; C12N 5/08; C12N 15/86; C12N 15/74

(52) U.S. Cl. 424/200.1; 435/472; 435/252.3; 435/456; 435/366; 800/14

(57) ABSTRACT

The present invention relates to a method for regulating the expression of a target gene in a prokaryotic cell and a horrigent suitable for conducting the method.

The present invention relates to a method for regulating the expression of a target gene in a prokaryotic cell and a reagent suitable for conducting the method.

RNA interference (RNAi) has been described in a plurality of different eukaryotic organisms, e.g. Caenorhabditis elegans and Drosophila as well as in various mammalian, e.g. human cells and in mammalian organisms. For example it is referred to PCT/EP01/13968, PCT/EP02/10881, PCT/EP03/05513, PCT/EP03/07516, EP 03 001059.9 and EP 03 001058.1 which are herein incorporated by reference.

Prokaryotic organisms encompass some of the major human pathogens, e.g. Mycobacterium tuberculosis, Salmonella typhimurium, Shigella sp., Staphylococcus aureus, Chlamydia pneumoniae and Clostridium diphtheriae. The genomes and genes of most of these pathogens have been described, however, functions could only be assigned to a small fraction of the identified genes so far. In general, knock-out strategies are used to identify genes which are associated with virulence, infectivity, toxicity and/or replication and which therefore represent highly potent drug targets. However, present gene knock-out strategies in prokaryotic organisms are expensive, time-consuming and are not suitable for high-throughput target validation. On the other hand, the most powerful gene knock-down technique, RNAi, has not been successful in prokaryotes so far. A method to induce RNAi in prokaryotic organisms would represent an extremely useful tool in order to validate and understand prokaryotic gene function and to identify novel drug targets. Further, RNA interference may also be used directly as therapeutic approach in prokaryotes.

Surprisingly, it was found that RNAi may be induced in prokaryotic cells by introducing into a prokaryotic cell a first component which is a RNAi compound or a precursor thereof, or a DNA molecule encoding a RNAi compound or a precursor thereof, and optionally a second component comprising compounds obtainable from eukaryotic cells, further prokaryotic cells or synthetic compounds. The term “RNAi compound” in this context relates to any molecule which is capable of inducing RNA silencing, i.e. transcriptional gene silencing or posttranscriptional gene silencing, particularly RNAi under suitable conditions in a prokaryotic cell, particularly in the presence of a second component as specified in detail below. Together, the first and the second components can induce a sequence-specific regulation of target gene expression in a prokaryotic cell. The first component also may suffice to induce RNA silencing, particularly RNAi in prokaryotic cells.

Thus, the present invention generally relates to a method for regulating the expression of a target gene in a prokaryotic cell as well as the use of this method e.g. in a functional gene and target validation, and diagnostic or therapeutic approaches.

A first aspect of the present invention relates to a method for regulating the expression of a target gene in a prokaryotic cell comprising the steps
(a) introducing into the prokaryotic cell a first component selected from
   (i) a RNA molecule capable of sequence-specific regulating the target gene expression,
   (ii) a RNA precursor molecule of (i) or
   (iii) a DNA molecule encoding the RNA molecule of (i) or (ii) and
(b) optionally introducing into the prokaryotic cell a second component selected from compounds obtainable from eukaryotic cells, further prokaryotic cells or synthetic compounds,
   wherein the first component optionally together with the second component is capable of inducing a sequence-specific regulation of the target gene expression.

Preferably, the method of the present invention comprises a regulation of the target gene expression by RNA silencing, particularly RNA interference, more preferably, the regulation of target gene expression is carried out by processes mediated by siRNA molecules. siRNA (small interfering RNA) molecules are described by Elbashir et al., 2001, Nature 411, 494-298.

In order to achieve sequence-specific target gene regulation, the RNA molecule (i) should exhibit a sufficient degree of sequence identity and/or sequence complementarity to a target RNA molecule, particularly an expression product of the target gene within the prokaryotic cell. Preferably, the sequence identity or complementarity is at least 85%, more preferably at least 90%, yet more preferably at least 95% and most preferably 100% in the portion of the RNA molecule (i) which is capable of sequence-specific gene regulation.

Within the present invention the term “complementarity” refers to the degree of similarity of two nucleic acid sequences with regard to Watson Crick and Non-Watson Crick base pairings.

In a first preferred embodiment, the RNA molecule (i) comprises a double-stranded (ds)RNA molecule, wherein each strand has a length of 15-30, preferably of 19-25 nucleotides. The dsRNA molecule may be blunt-ended. It is, however, preferred that at least one RNA strand comprises a 3′-overhang of 1-5, preferably 1-3 nucleotides. The 3′-overhangs are not necessarily identical or complementary to the target sequence and may be stabilized against degradation by modification, e.g. incorporation of purine nucleotides and/or replacement of pyrimidine nucleotides by modified nucleotide analogs and/or by incorporation of deoxyribonucleotides. Examples of suitable dsRNA molecules, e.g. siRNA molecules are described in PCT/EP 01/13968, PCT/EP 03/05513 and EP 03001059.9.

In a further embodiment, single-stranded (ss)RNA molecules (i) having a length of 15-60, particularly of 19-50 nucleotides are employed. Preferably, these ssRNA molecules are capable of forming a secondary structure by internal base pairing. Examples for suitable ssRNA molecules are disclosed in PCT/EP 03/07516.

The RNA molecules (i) may comprise at least one modified nucleotide analog. The nucleotide analogs are preferably at positions where the activity is not substantially impaired as for example at a region at the 5′-end and/or at the 3′-end of the RNA molecule (i). Especially overhangs can be stabilized by inserting modified nucleotide analogs. Preferably, the RNA molecules contain at least 50%, preferably at least 75% non-modified ribonucleotide units. Furthermore, the RNA molecules (i) do not contain more than 8, especially preferred not more than 4 deoxyribonucleotide units.

Preferred nucleotide analogs are selected from sugar or backbone-modified ribonucleotides but also ribonucleotides having nucleobases which are not naturally occurring, instead of a naturally occurring nucleobase. Examples for such non-naturally occurring nucleobases are uridine or cytidine analogs, modified at position 5, e.g. 5-(2-amino)propyluridine or 5-bromo-uridine, adenosine or guanosine analogs, modified at position 8, e.g. 8-bromo-guanosine, deazanucleotides, e.g. 7-deazaadenosine, O- and N-alkylated nucleobases, e.g. N6-methyladenosine. In sugar-modified ribonucleotides, the 2′OH-group is preferably replaced by a group selected from H, OR, R, halo, SH, SR, NH₂, NHR, NR₂ or CN, wherein R is C₁-C₆ alkyl, C₂-C₆ alkenyl or C₂-C₆alkynyl, and halo is F, Cl, Br or I. In preferred backbone modified ribonucleotides, the phosphoester group linking two adjacent ribonucleotides is modified, e.g. replaced by a phosphothioate group. It has to be noted that one or more of the above-mentioned modifications can be combined with each other.

In a further embodiment, a RNA precursor molecule (ii) of a RNA molecule (i) may be employed. The term “RNA precursor molecule” is well known in the art and refers to any RNA species that is not yet the mature RNA product and which may include a 5′ clipped region (5′ clip), a 5′ untranslated region (5′ UTR), coding sequences (CDS, exon), intervening sequences (intron), a 3′ untranslated region (3′ UTR), and a 3′ clipped region (3′ clip) (see DDBJ/EMBL/Genbank Feature Table: Definition; http://www.ncbi.nlm.nih.gov/projects/Collab/FT/). Preferably, RNA precursor molecules are chosen such that active RNA molecules (i) are generated by processing mechanisms employing compounds within the prokaryotic cell and/or mediated by the co-introduced second component. Compounds involved in processing of RNA precursors include enzymes (proteins and/or ribozymes) which generate small single-stranded or double-stranded RNA molecules capable of inducing RNA silencing.

Such enzymes or components thereof can be of prokaryotic or eukaryotic origin, can be included in extracts prepared from prokaryotic or eukaryotic cells, can be recombinantly expressed, or can be chemically synthesized. These compounds can be components of the Drosha, Dicer or RISC complexes.

Further, RNA precursor molecules may be chosen such that active RNA molecules (i) are generated by RNA replication processes, e.g. mediated by a RNA-dependent RNA polymerase such as Qγβ polymerase.

In yet a further embodiment of the present invention, the RNA molecule (i) is generated within the prokaryotic cell by expression of a DNA molecule which encodes the RNA molecule (i) or an precursor thereof. The DNA molecule preferably comprises an expression control sequence, e.g. a promoter sequence optionally in combination with operator, repressor, and/or enhancer sequences, which is transcriptionally active in the prokaryotic cell, in operative linkage to a sequence encoding the RNA molecule (i) or the RNA precursor molecule (ii). If the RNA molecule (i) is a double-stranded molecule, the DNA molecule (iii) may comprise two sequences each coding for a strand of the double-stranded RNA molecule in operative linkage with a single expression control sequence or alternatively in operative linkage with different expression control sequences. The DNA molecule (iii) may be present on a vector, e.g. an episomal vector, particularly a plasmid, or may be present on a vector, which may be integrated into the chromosome of the cell such as a viral vector. Further, the RNA precursor molecule (ii) or the DNA molecule (iii) encoding the RNA molecule (i) or the RNA precursor molecule (ii) may be present on a bacteriophage which is capable of infecting the respective host cell and/or the prokaryotic target cell.

The method of the present invention comprises introducing into the prokaryotic cell a first component, i.e. nucleic acid molecule (i), (ii) or (iii) and optionally a second component comprising compounds obtainable from eukaryotic cells, prokaryotic cells or synthetic compounds. The first component is a nucleic acid which may be introduced into the prokaryotic cell according to any suitable procedure known in the art, e.g. by CaCl₂ or RbCl transformation, by electroporation etc. The second component is preferably introduced by electroporation or any other suitable procedure. Both components may be co-introduced simultaneously, e.g. by electroporation. It should be noted, however, that both components may be introduced into the prokaryotic cell at different times. For example, the prokaryotic cell may be transformed with the nucleic acid molecule (i), (ii) or (iii) by any suitable method and subsequently the second component may be introduced. It is however also possible that the second component is introduced before the first component.

The second component comprises compounds obtainable from eukaryotic or further prokaryotic cells which are capable, together with the first component, of inducing a sequence-specific regulation, e.g. inhibition of target gene expression in a prokaryotic cell. The second component may comprise a cell extract, a cell extract fraction or purified components from a cell extract. For example, the composition may comprise a cell extract or soluble components thereof, which may be obtained by freeze-thaw-lysis and/or any other suitable procedure, such as by a shear treatment, e.g. by pushing/pulling the cells through the needle of a syringe. The second component may be selected from naturally occurring products such as RISC (RNA-induced silencing complex) components and recombinant products derived from eukaryotes or further prokaryotes. RISC is a complex that regulates gene expression at many levels, comprising a number of the Argonaute (Ago) family of proteins, as defined by the presence of PAZ and PIWI domains. Known or apparent components include the B or R2 complex of D. melanogaster embryos, Dcr1+2, R2D2. Ago2, AGO1+2, Fmr1/Fxr, Tsn, Vig, L5, L11, 5S rRNA, Dmp68, Gemin 3 and Gemin 4, as well as other, not yet clearly identified components. A non-limiting list of RISC components can be found in the review of Eric J. Sontheimer: Assembly and functions of RNA silencing complexes (2005). Nature Review Molecular Cell Biology 6, 127-138 or in G. Meister and T. Tuschl, Mechanisms of gene silencing by double-stranded RNA (2004). Nature 431, 343-349, which is hereby incorporated by reference.

The eukaryotic cell may be an animal cell, a protist cell, a plant cell or a fungal cell, e.g. yeast cell. Preferably, the cell is a mammalian cell such as a human cell, e.g. a Hela cell or an NIH3T3 cell, an insect cell, e.g. a Drosophila cell, a nematode cell, e.g. a Caenorhabditis elegans cell or a plant cell.

The further prokaryotic cell is selected from a prokaryotic cell of another species or strain which is different from the prokaryotic cell in which a target gene is to be regulated (prokaryotic target cell), or a recombinant strain, a mutant or a transformed variant of the prokaryotic target cell.

The second component may also comprise at least one synthetic compound which can be a protein, a peptide, a nucleic acid, a peptide nucleic acid (PNA), a lipid, a carbohydrate, a low molecular weight compound such as an amino acid or a nucleotide which is analogue or not in sequence and/or structure to natural compounds or a combination of any of these compounds.

The method of the present invention allows the regulation of the expression of a target gene within a prokaryotic cell. The target gene is preferably a gene which is located on the chromosome of a prokaryotic cell. It should be noted however, that also episomal target genes, e.g. target genes, which are located on a extrachromosomal vector, e.g. a plasmid may be regulated by the method of the invention. The present invention allows the regulation of a single target gene or plurality of target genes, e.g. by introducing several different nucleic acid molecules (i), (ii) and/or (iii), which are directed to different target sequences.

Inhibition of episomal gene expression is lasting and the inhibited clones remain silent. This inhibition does not necessarily affect all genes on the episome, however (see e.g. FIG. 8, in which inhibition of episomal gene expression is specific and in which antibiotics resistance remains unaffected).

Inhibition of chromosomal gene expression is at least transient, eventually also lasting.

A further aspect of the present invention relates to a prokaryotic cell which is transformed with a first component selected from
(i) a RNA molecule capable of sequence-specific regulating the target expression of a target gene sequence within the prokaryotic cell,
(ii) a RNA precursor molecule of (i) or
(iii) a DNA molecule encoding the RNA molecule of (i) or (ii).

The prokaryotic cell is preferably further transformed with a second component comprising compounds obtainable from eukaryotic cells, further prokaryotic cells or synthetic compounds capable of inducing a sequence-specific regulation of the target gene expression, together with the first component.

The prokaryotic cell may be an archaea cell, a bacteria cell including gram-positive, gram-negative and mycobacteria, or a cell of phylogenetically unaffiliated bacteria. For example, the prokaryotic cell may be a cell from a laboratory strain, e.g. E. coli or B. subtilis. On the other hand, the prokaryotic cell may be a cell from a pathogenic strain, e.g. a Mycobacterium cell, a Salmonella cell etc.

Still a further aspect of the present invention relates to a reagent composition or kit for regulating the expression of a target gene in a prokaryotic cell comprising a first component selected from
(a) a RNA molecule capable of sequence-specific regulating the expression of a target gene sequence within the prokaryotic cell,
   (ii) a RNA precursor molecule of (i) or
   (iii) a DNA molecule encoding the RNA molecule of (i) or (ii) and
(b) optionally a second component comprising compounds obtainable from eukaryotic cells, further prokaryotic cells or synthetic compounds capable of inducing a sequence-specific regulation of the target gene expression, together with the first component.

Components (a) and (b) of the reagent composition or kit may be provided as a mixture or as separate reagents.

Still a further aspect of the present invention relates to an eukaryotic cell or a non-human eukaryotic organism infected with a prokaryotic cell of the present invention as described above. Examples of suitable eukaryotic cells are animal cells including human cells, plant cells and fungal cells as described above. Examples of suitable non-human eukaryotic organisms are all kinds of laboratory and useful animals, e.g. mice, rats, primates etc. as well as all kinds of laboratory and useful plants. The infected cells or organisms may be used for the assessment of gene function, particularly for the identification and/or characterization of prokaryotic gene function.

Still a further aspect of the present invention relates to the use of a RNAi compound selected from
(i) a RNA molecule capable of sequence-specific regulating the expression of a target gene,
(ii) a RNA precursor molecule of (i) or
(iii) a DNA molecule encoding the RNA molecule of (i) or (ii),
   for the manufacture of a diagnostic or therapeutic agent for monitoring and/or modulating the expression of a target gene in a prokaryotic cell.

More particularly, the RNAi compound is suitable for the manufacture of a therapeutic agent for targeting and suppressing prokaryotic gene expression and replication in human and non-human eukaryotic organisms infected with a prokaryotic cell of the present invention as described above in order to defend against Actinomycosis, Anthrax, Aspergillosis, Bacteremia, Bartonella Infections, Botulism, Brucellosis, Burkholderia Infections, Campylobacter Infections, Candidiasis, Cat-Scratch Disease, Chlamydia Infections, Cholera, Clostridium Infections, Coccidioidomycosis, Cryptococcosis, Dermatomycoses, Diphtheria, Ehrlichiosis, Escherichia coli Infections, Fasciitis, Necrotising Infections, Fusobacterium Infections, Gas Gangrene, Histoplasmosis, Impetigo, Klebsiella Infections, Legionellosis, Leprosy, Leptospirosis, Listeria Infections, Lyme Disease, Maduromycosis, Melioidosis, Mycobacterium Infections, Mycoplasma Infections, Mycoses, Nocardia Infections, Onychomycosis, Ornithosis, Plague, Pneumococcal Infections, Pseudomonas Infections, Q Fever, Rat-Bite Fever, Relapsing Fever, Rheumatic Fever, Rickettsia Infections, Rocky Mountain Spotted Fever, Salmonella Infections, Scarlet Fever, Scrub Typhus, Sepsis, Staphylococcal Infections, Streptococcal Infections, Tetanus, Tick-Borne Diseases, Tuberculosis, Tularemia, Typhoid Fever, Typhus, Epidemic Louse-Borne Infections, Vibrio Infections, Yaws, Yersinia Infections, Zoonoses, and Zygomycosis.

The present invention allows an induction of RNAi prokaryotic organisms and hence represents a powerful tool in order to investigate prokaryotic gene function and to identify or characterize target genes for diagnostic or therapeutic approaches and/or to identify or characterize pharmaceutical agents e.g. in a high-throughput compatible manner. RNA molecules may also be used as antibacterial drugs in a direct form either in vitro, ex vivo or in human and non-human organisms infected with a prokaryotic cell as described above.

It should be noted that all preferred embodiments discussed for one or several aspects of the invention also relate to all other aspects. This particularly refers to all features disclosed in the present invention regarding the RNA molecule (i), RNA precursor molecule (ii) and the DNA molecule encoding the RNA molecule of (i) or (ii) and the compounds of the second component.

The following figures and examples illustrate the invention and are non-limiting embodiments of the invention as claimed below. Numerous additional aspects and advantages of the invention will become apparent to those skilled in the art upon consideration of the following description of the figures which describes presently preferred embodiments thereof.

Further, the present invention is illustrated in more detail by the following Figures and Examples.

### DESCRIPTION OF FIGURES

FIG. 1

Suppression of episomal EGFP expression by RNAi in Mycobacterium smegmatis. EGFP coding plasmid DNA was delivered alone (/), together with EGFP-directed siRNA (GFP-siRNA) or together with a control duplex (control-siRNA) via electroporation (EP) into the bacterial cells. Nucleic acids were pre-treated or not treated with varying amounts of eukaryotic compounds. A) Kinetics of EGFP expression. Specific siRNA-mediated gene knock down was only observed in the presence of undiluted eukaryotic compounds. Averages of two EP experiments. B) Bacterial proliferation in selective medium at day 7 post EP. A slight co-suppression of the kanamycin gene was observed in the presence of GFP-siRNA and eukaryotic compounds.

FIG. 2

Suppression of episomal EGFP expression by RNAi in Mycobacterium smegmatis. EGFP coding plasmid DNA was delivered alone (/), together with EGFP-directed siRNA (GFP-siRNA) or together with a control duplex (control-siRNA) via electroporation (EP) into the bacterial cells. Nucleic acids were pre-treated or not treated with eukaryotic compounds. A) Kinetics of EGFP expression. Specific siRNA-mediated gene knock-down was transient and could be observed until day 4 post EP. Average of five EP experiments. B) EGFP expression at day 3 post EP. siRNA-mediated gene suppression was only observed in the presence of eukaryotic compounds. Average of five EP experiments. C) Bacterial replication in selective medium one day post EP. Co-suppression of the kanamycin gene was observed in the presence of GFP-siRNA and eukaryotic compounds. A general lower proliferation was observed in the presence of eukaryotic compounds.

FIG. 3

Suppression of episomal EGFP expression by RNAi in Mycobacterium smegmatis. EGFP coding plasmid DNA was delivered alone (no RNA), together with EGFP-directed siRNA (GFP-siRNA) or together with a control duplex (control-siRNA) via EP into the bacterial cells. Nucleic acids were pre-treated (left panel) or not treated (right panel) with eukaryotic compounds. Mixed cultures of five independent EP experiments. Bacteria were plated on selective agar directly after transformation. Pictures show EGFP expression (520 nm) after excitation (485 nm) and corresponding bacterial growth (phase contrast). Specific GFP-siRNA-mediated gene suppression of EGFP expression as well as strong co-suppression of the kanamycin gene was observed in the presence of GFP-siRNA and eukaryolic compounds. Generally, a reduced bacterial growth was observed in the presence of eukaryotic compounds.

FIG. 4

Suppression of episomal EGFP expression by RNAi in Mycobacterium smegmatis. EGFP coding plasmid DNA was delivered alone (no RNA), together with EGFP-directed siRNA (GFP-siRNA) or together with a control duplex (control-siRNA) via EP into the bacterial cells. Nucleic acids were pre-treated (left panel) or not treated (right panel) with eukaryotic compounds. Mixed cultures of five independent EP experiments. Bacteria were plated at day 3 post transformation on selective agar. Pictures show EGFP expression (520 nm) after excitation (485 nm) and corresponding bacterial growth (phase contrast). Specific GFP-siRNA-mediated gene suppression of EGFP expression as well as strong co-suppression of the kanamycin gene was observed in the presence of GFP-siRNA and eukaryotic compounds. Generally, a reduced bacterial growth was observed in the presence of eukaryotic compounds.

FIG. 5

Suppression of constitutive (genomic) GFP expression in Salmonella typhimurium by RNAi. Eukaryotic compounds were delivered alone (no RNA), together with EGFP-directed siRNA (GFP-siRNA) or together with a control duplex (control-siRNA) via EP into the bacterial cells. A) Kinetics of EGFP expression. Specific siRNA-mediated gene knock-down was only observed in the presence of siRNA and eukaryotic compounds at days 2 and 3 post EP. Due to high basal expression levels and the long half live of EGFP, suppression of constitutive EGFP expression was less pronounced but significant compared to de novo expression from episomal DNA. Average of five EP experiments. B) Bacterial proliferation in selective medium detected one day post EP. There is no evidence for a co-suppressive effect on non-target genes.

FIG. 6

Suppression of constitutive (genomic) GFP expression in Escherichia coli by RNAi. Eukaryotic compounds were delivered alone (no RNA), together with EGFP-directed siRNA (GFP-siRNA) or together with a control duplex (control siRNA) via EP into the bacterial cells. Averages of 2 independent experiments of each 5 EPs per sample. A) Kinetics of EGFP expression. Specific siRNA-mediated gene knock down was observed only in the presence of siRNA and eukaryotic compounds. B) Kinetics of EGFP expression relative to cells not transfected with RNA.

FIG. 7

Knock-down of episomal GFP expression in Listeria monocytogenes by RNAi. Eukaryotic compounds were delivered alone (no RNA), together with EGFP-directed siRNA (GFP-siRNA) or together with a control duplex (control siRNA) via EP into the bacterial cells. A) Kinetics of EGFP expression. B) Comparison of EGFP expression (left panel) and bacterial growth (right panel) at day 11 post EP. Profound siRNA-mediated EGFP suppression did not affect bacterial growth.

FIG. 8

Suppression of episomal EGFP expression by RNAi in Listeria monocytogenes (L. monocytogenes). Eukaryotic compounds were delivered alone (no RNA), together with GFP-specific siRNA (GFP-siRNA), and Control siRNA (Control-siRNA) via EP into electrocompetent cells of L. monocytogenes carrying an EGFP-expressing plasmid. Gene silencing resulted in completely and permanently silenced bacterial cells and clones. Silenced clones were isolated and re-cultured over a period of three month. All clones remained silent during the period of observation. Furthermore, no co-suppression of other episomal located genes, i.e. the gene mediating antibiotics resistance, was observed. Left-hand side: fluorescence photographs of bacterial cultures; right-hand side: fluorescence and phase contrast photographs of monoclonal colonies.

FIG. 9

Suppression of Bacillus anthracis (B. anthracis) lethal factor (LF) expression by RNAi. In B. anthracis, LF is located on the naturally occurring plasmid pXO1. Electrocompetent cells of B. anthracis strain Sterne A15 (pXO1+, pXO2-) were electroporated with buffer, LF-specific siRNA or control siRNA in the presence of eukaryotic cell extracts. LF expression was detected in the culture medium by ELISA using a mouse monoclonal anti-LF antibody (ab), a biotinylated polyclonal goat anti-mouse IgG ab, and alkaline phosphatase-coupled streptavidin.

### EXAMPLE

### Regulation of Gene Expression in Prokaryotic Organisms

1. Selection of Target Sequences and RNAi Compounds

Double stranded siRNA molecules consisting of a sense and an antisense strand directed against target sequences from the GFP and luciferase gene, as well as the B. anthracis lethal factor-directed sequence, were manufactured by solid phase synthesis according to standard protocols. The two desoxynucleotides at the 3%-end of the RNA SEQ ID NOs:5, 6, 8, 9, 11 and 12 are not shown in the sequence listing.

GFP-Directed Sequence:

| | |
|---|---|
| target: | |
| 5′-CGGCAAGCTGACCCTGAAGTTCAT-3′ | (SEQ ID NO:1) |
| sense: | |
| 5′-GCAAGCUGACCCUGAAGUUCAU-3′ | (SEQ ID NO:2) |
| antisense: | |
| 5′-GAACUUCAGGGUCAGCUUGCCG-3′ | (SEQ ID NO:3) |

Luciferase-Directed Control Sequences:

| | |
|---|---|
| target: | |
| 5′-AACATCACGTACGCGGAATACTT-3′ | (SEQ ID NO: 4) |
| sense: | |
| 5′-CAUCACGUACGCGGAAUAACdTdT-3′ | (SEQ ID NO: 5) |
| antisense: | |
| 5′-GUAUUCCGCGUACGUGAUGdTdT-3′ | (SEQ ID NO: 6) |
| target: | |
| 5′-AACGTACGCGGAATACTTCGA-3′ | (SEQ ID NO: 7) |
| sense: | |
| 5′-CGUACGCGGAAUACUUCGAdTdT-3′ | (SEQ ID NO: 8) |
| antisense: | |
| 5′-UCGAAGUAUUCCGCGUACGdTdT-3′ | (SEQ ID NO: 9) |

B. Anthracis Lethal Factor-Directed Sequence:

| | |
|---|---|
| target: | |
| 5′-CAUCAAUCCAUUGGAAGUACCUU-3′ | (SEQ ID NO: 10) |
| sense: | |
| 5′-UCAAUCCAUUGGAAGUACCdTdT-3′ | (SEQ ID NO: 11) |
| antisense: | |
| 5′-GGUACUUCCAAUGGAUUGAdTdG-3′ | (SEQ ID NO: 12) |

2. Production of Cell Extracts

Each 3 confluent 175 cm² tissue culture flasks of HeLa and NIH 3T3 cells (each approximately 2×10⁷ cells) were trypsinized, washed three times with PBS, sedimented and resuspended in 700 μl PBS. 3 different protease inhibitors were added; half of the resuspended cells were frozen and thawed 4 times, the second half of cells were pushed and pulled 20× through a thin needle of a syringe, two fractions were mixed again and centrifuged at maximum speed for 10 min. The supernatant was frozen in liquid nitrogen in aliquots, which were freshly thawed prior to any experiment and which were never frozen back.

3. Transformation of Prokaryotic Cells

Electro-competent prokaryotic cells were prepared following standard protocols. Briefly, bacteria were grown in appropriate culture medium to OD₆₀₀=0.2 to 0.4, washed 4 to 5 times with 10-15% glycerol or HEPES/sucrose, taken up in the wash buffer, frozen in liquid nitrogen and stored at −80° C.

100 pmol siRNA and 1 μl cell extract were incubated in 25 μl water or HEPES buffer at room temperature for 5 min. In order to target episomal target gene expression 1 μg of plasmid DNA carrying the target gene were co-incubated as well. Then, 100 μl pre-cooled electro-competent bacteria were added and 100 μl of this mixture were used for electroporation in pre-cooled 0.2 cm cuvettes. Electroporation was performed at 2.5 kV voltage and 25 μFd capacitance. Resistance was 200Ω for Salmonella and Bacillus anthracis, 400Ω for E. coli and Listeria, and 1000Ω for Mycobacteria. Bacteria were placed on ice for 2 min, taken up in 1 ml culture medium and placed on a shaker at 37° C. or partly plated on agar. Once a day EGFP expression was monitored from 500 μl of the cultures using a Fluoroscan (ascent). Alternatively, cells growing on agar were visualized by fluorescence microscopy.

4. Results

siRNA with homology to the GFP reporter gene as well as unspecific control RNA were delivered via electroporation into different prokaryotic target cells. As target cells we used a wild type strain of Mycobacterium smegmatis (M. smegmatis) and a transformed wild type strain of Listeria monocytogenes (L. monocytogenes) carrying a plasmid containing the GFP gene, and recombinant Salmonella typhimurium (S. typhimurium) and Escherichia coli (E. coli) strains carrying a chromosomally integrated GFP gene.

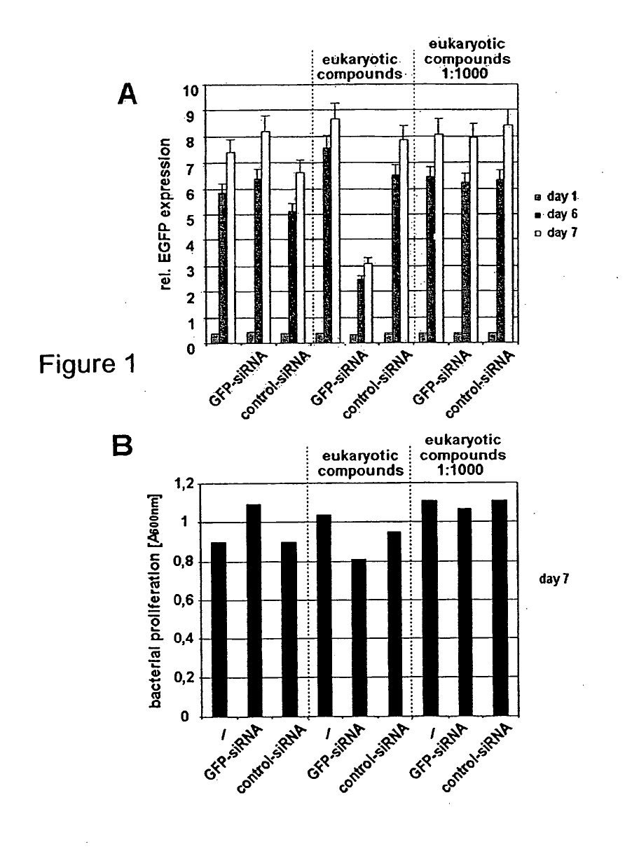

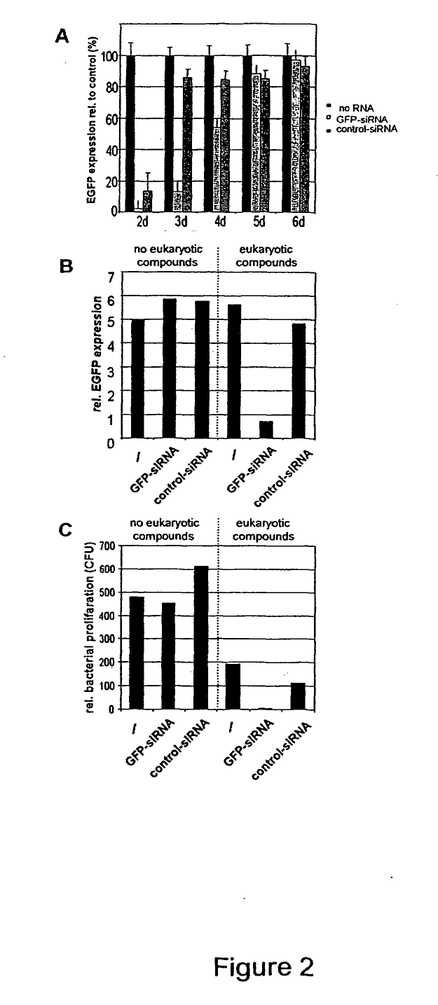

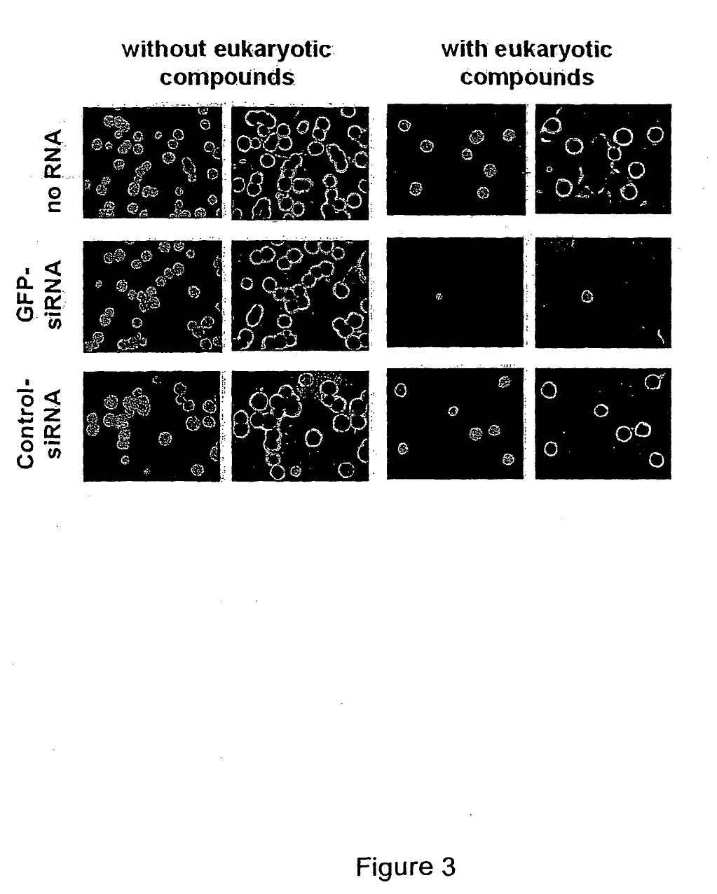

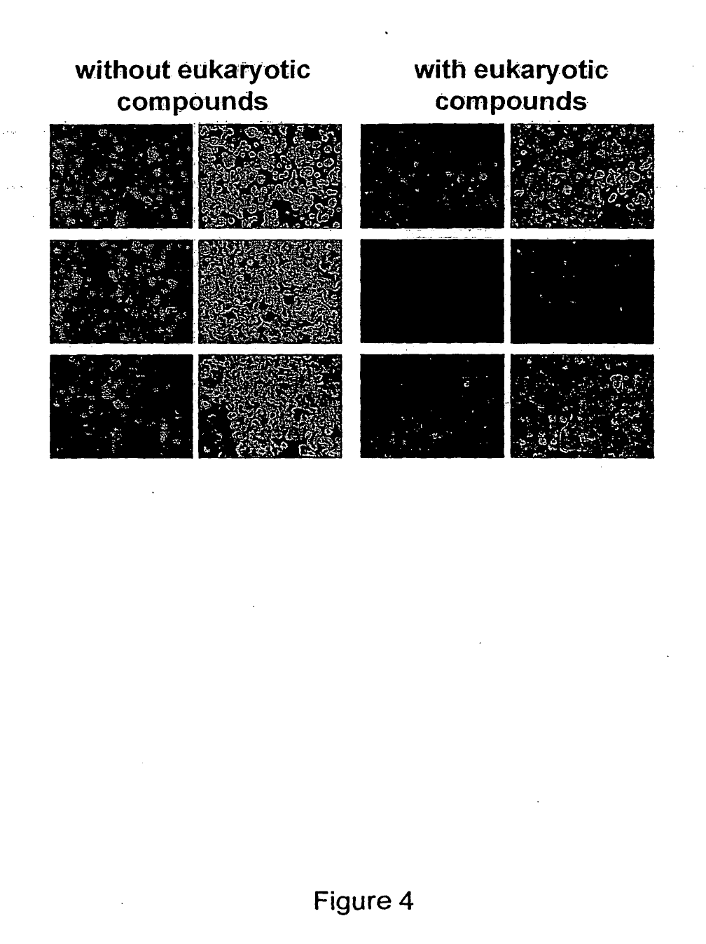

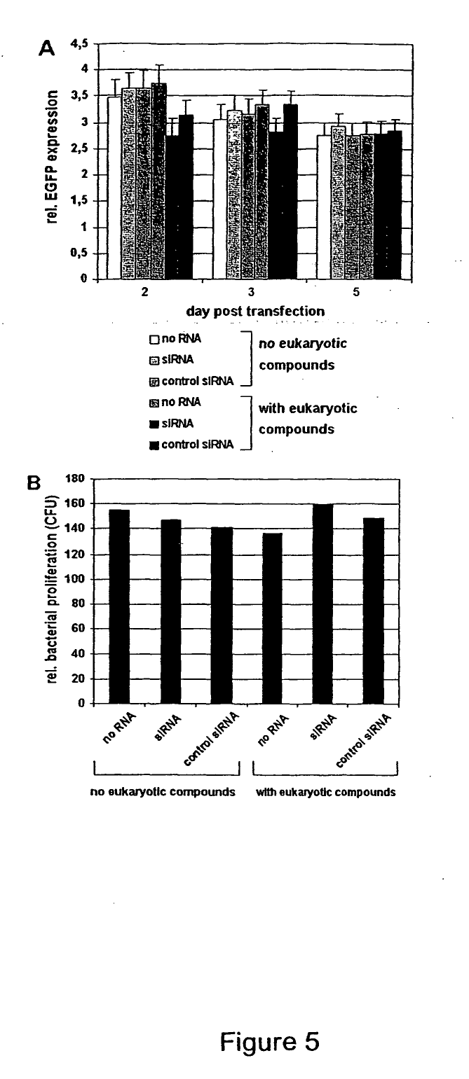

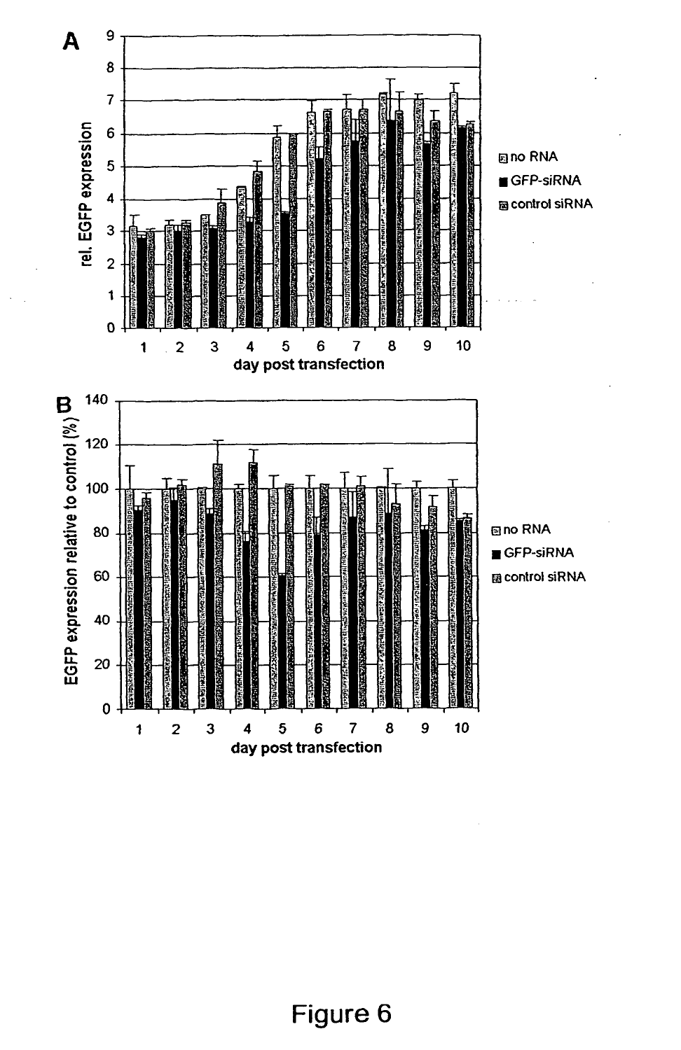

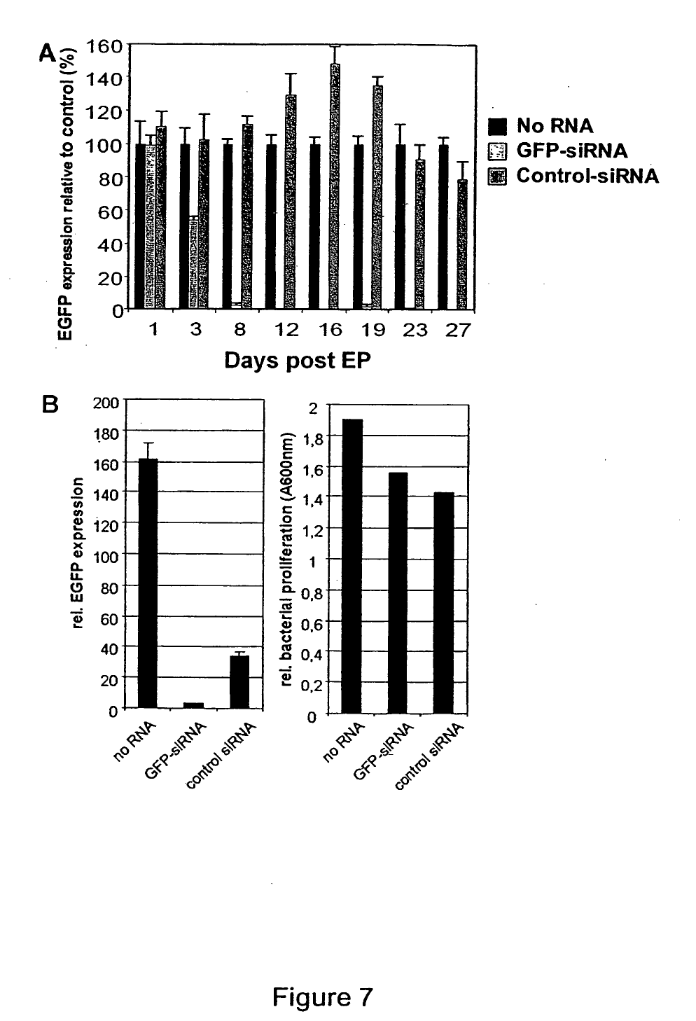

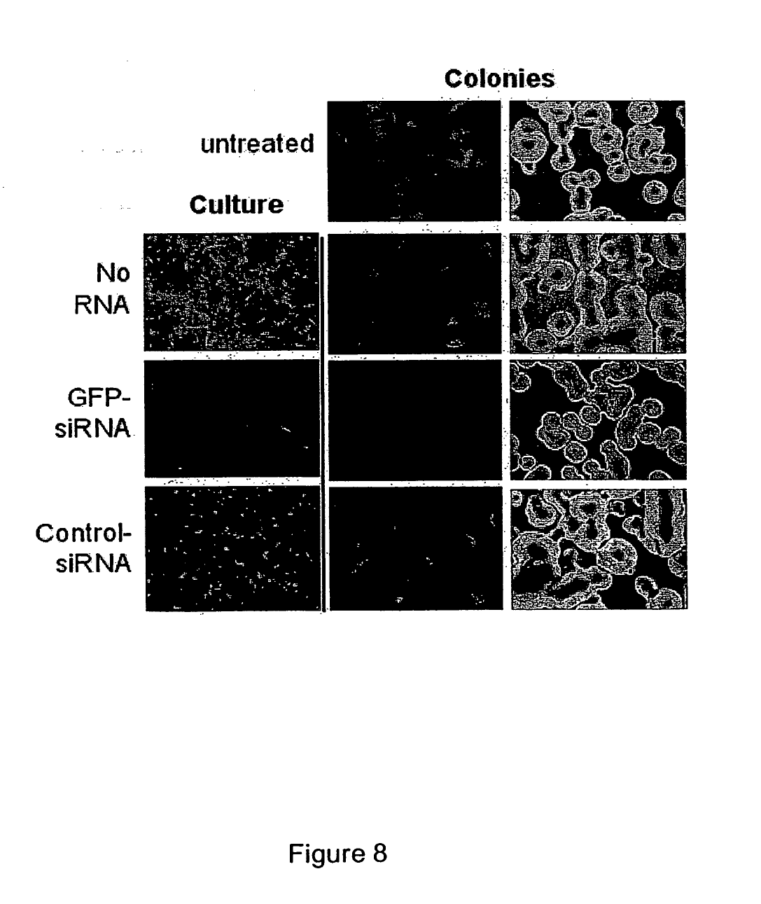

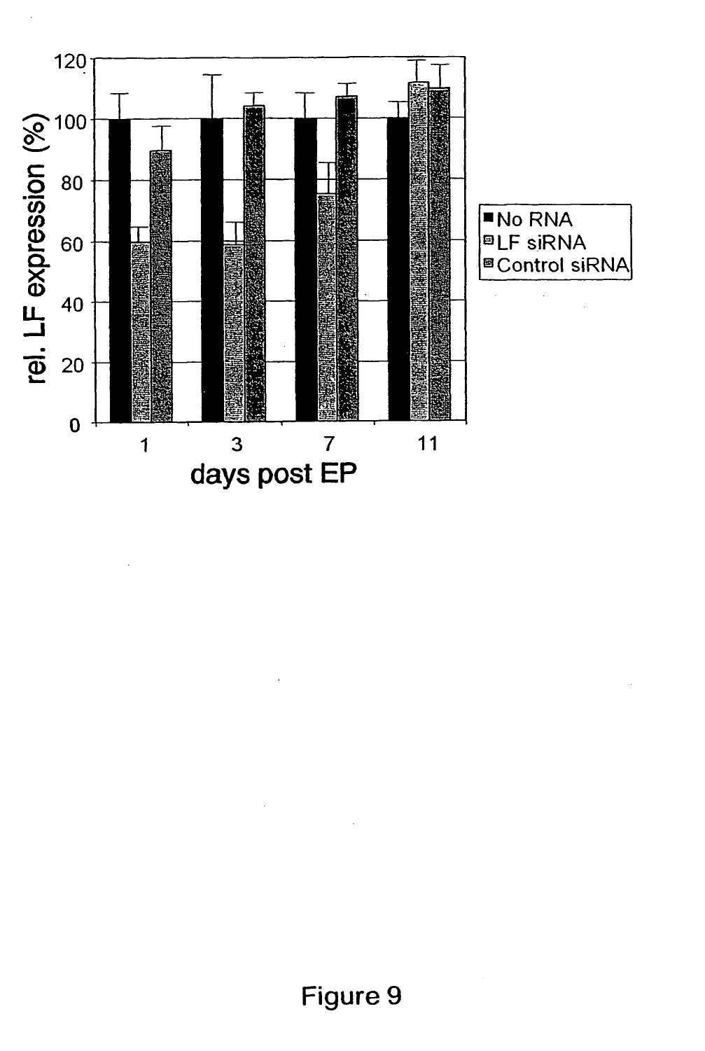

In case of M. smegmatis, an episomal GFP gene was co-delivered together with the RNA during electroporation. The delivery of naked siRNA did not result in the induction of siRNA-mediated gene suppression (RNAi), indicating that RNAi seems not to be an intrinsic mechanism in prokaryotes. However, it cannot be excluded, that RNAi might be induced by RNAi compounds or siRNA alone but needs optimized procedures to be detectable. Sequence-specific induction of RNAi was observed upon delivery of siRNA pre-treated with compounds derived from eukaryotic cells (FIGS. 1-9). Therefore, the method of this invention concerns the co-provision of siRNA and eukaryotic compounds to prokaryotic cells in order to successfully induce RNAi.

In case of M. smegmatis a co-suppressive effect was observed that concerned non-target genes (here: antibiotic resistance genes) located on the same episome (FIGS. 1B, 2C). In case of all other used prokaryotes, induction of RNAi by the method of this invention did not affect bacterial growth, indicating that siRNA-mediated gene suppression is target-specific (FIG. 5).

1. A method for regulating the expression of a target gene in a prokaryotic cell comprising the steps
(a) introducing into the prokaryotic cell a first component selected from
(i) a RNA molecule capable of sequence-specific regulating the target gene expression, having at least 85% sequence complementarity to a target gene within said prokaryotic cell
(ii) a RNA precursor molecule of (i) or
(iii) a DNA molecule encoding the RNA molecule of (i) or
(ii) and
(b) introducing into said prokaryotic cell a second component selected from compounds obtainable from eukaryotic cells, further prokaryotic cells or synthetic compounds,
wherein the first component together with the second component is capable of inducing a sequence-specific regulation of the target gene expression.

2. The method of claim 1 wherein the target gene expression is regulated by RNA silencing, i.e. transcriptional gene silencing or posttranscriptional gene silencing.

3. The method of claim 1 wherein the target gene expression is regulated by RNA interference.

4. The method of claim 1 wherein the RNA molecule (i) is a double-stranded RNA molecule wherein each strand has a length of 15-30, preferably 19-25 nucleotides.

5. The method of claim 4 wherein at least one strand of the double-stranded RNA molecule has a 3′ overhang of 1-5, preferably of 1-3 nucleotides.

6. The method of claim 5 wherein the 3′-overhang is stabilized against degradation.

7. The method of claim 1 wherein the RNA molecule (i) is a single-stranded RNA molecule having a length of 15-60, particularly 19-50 nucleotides.

8. The method of claim 1 wherein the RNA molecule (i) comprises at least one modified nucleotide analog and/or deoxyribonucleotide.

9. The method of claim 1 wherein said RNA precursor molecule (ii) is processed to the active RNA molecule (i) by compounds present within the prokaryotic cell and/or in the second component.

10. The method of claim 1 wherein DNA molecule (iii) comprises an expression control sequence in operative linkage to a sequence encoding the RNA molecule (i) or (ii).

11. The method of claim 1 wherein the DNA molecule (iii) is located on a vector.

12. The method of claim 10 wherein the vector is selected from plasmids, viral vectors and bacteriophages.

13. The method of claim 1 wherein steps (a) and (b) are carried out simultaneously.

14. The method of claim 1 wherein steps (a) and (b) are carried out subsequently.

15. The method of claim 1 wherein step (a) and/or step (b) comprises an electroporation.

16. The method of claim 1 wherein the second component comprises an eukaryotic cell extract, an eukaryotic cell extract fraction or purified components from an eukaryotic cell extract, a prokaryotic cell extract, a prokaryotic cell extract fraction, purified components from prokaryotic cell extract or synthetic compounds.

17. The method of claim 16 wherein the eukaryotic or prokaryotic cell extract is obtained by freeze-thaw-lysis and/or shearing treatment of an eukaryotic or prokaryotic cell.

18. The method of claim 1 wherein the eukaryotic cell is selected from animal cells, protist cells, plant cells and fungal cells.

19. The method of claim 18 wherein the eukaryotic cell is a mammalian cell, e.g. a human cell.

20. A prokaryotic cell which is transformed with a first component selected from
(i) a RNA molecule capable of sequence-specific regulating the expression of a target gene sequence within the prokaryotic cell, having at least 85% sequence complementarity to a target gene within said prokaryotic cell,
(ii) a RNA precursor molecule of (i) or
(iii) a DNA molecule encoding the RNA molecule of (i) or (ii).

21. The cell of claim 20 which is further transformed with a second component comprising a compound obtainable from eukaryotic cells, further prokaryotic cells or synthetic compounds capable of inducing a sequence-specific regulation of the target gene expression together with the first component.

22. Reagent composition or kit for regulating the expression of a target gene in prokaryotic cell comprising
(a) a first component selected from a RNA molecule capable of sequence-specific regulating the expression of a target gene sequence within the prokaryotic cell, having at least 85% sequence complementarity to a target gene within said prokaryotic cell,
(ii) a RNA precursor molecule of (i) or
(iii) a DNA molecule encoding the RNA molecule of (i) or (ii) and
(b) a second component comprising compounds obtainable from eukaryotic cells, further prokaryotic cells or synthetic compounds capable of inducing a sequence-specific regulation of the target gene expression together with the first component.

23. An eukaryotic cell infected with a prokaryotic cell according to claim 20.

24. A non-human eukaryotic organism infected with a prokaryotic cell according to claim 20.

25. The organism of claim 24 which is an animal, a protist, a plant or a fungus.

26. The use of a cell or a non-human organism of claim 23, for the assessment of gene function.

27. The use of a RNA silencing compound selected from
(i) a RNA molecule capable of sequence-specific regulating the expression of a target gene, having at least 85% sequence complementarity to a target gene within said prokaryotic cell (ii) a RNA precursor molecule of (i) or
(iii) a DNA molecule encoding the RNA molecule of (i) or (ii) for modulating and/or monitoring the expression of a target gene in a prokaryotic cell.

28. The use of claim 27 for the manufacture of a therapeutic agent for treating a bacterial disease.

29. The use of claim 27 for the manufacture of a diagnostic agent for diagnosing a bacterial disease.


SEQUENCE LISTING (12 sequences)

SEQ ID NO: 1 - DNA, 24 bp, Artificial Sequence
  oligonucleotide seqeunce derived from green fluorescent protein
cggcaagctgaccctgaagttcat

SEQ ID NO: 2 - RNA, 22 nt, Artificial Sequence
  oligonucleotide with homology to green fluorescent protein
gcaagcugacccugaaguucau

SEQ ID NO: 3 - RNA, 22 nt, Artificial Sequence
  oligonucleotide with homology to green fluorescent protein
gaacuucagggucagcuugccg

SEQ ID NO: 4 - DNA, 23 bp, Artificial Sequence
  oligonucleotide seqeunce derived from luciferase
aacatcacgtacgcggaatactt

SEQ ID NO: 5 - DNA, 22 bp, Artificial Sequence
  oligonucleotide with homology to luciferase
  misc_feature (21)..(22): desoxynucleotide bases
caucacguacgcggaauaactt

SEQ ID NO: 6 - DNA, 21 bp, Artificial Sequence
  oligonucleotide with homology to luciferase
  misc_feature (20)..(21): desoxynucleotide bases
guauuccgcguacgugaugtt

SEQ ID NO: 7 - DNA, 21 bp, Artificial Sequence
  oligonucleotide seqeunce derived from luciferase
aacgtacgcggaatacttcga

SEQ ID NO: 8 - DNA, 21 bp, Artificial Sequence
  oligonucleotide with homology to luciferase
  misc_feature (20)..(21): desoxynucleotide bases
cguacgcggaauacuucgatt

SEQ ID NO: 9 - DNA, 21 bp, Artificial Sequence
  oligonucleotide with homology to luciferase
  misc_feature (20)..(21): desoxynucleotide bases
ucgaaguauuccgcguacgtt

SEQ ID NO: 10 - RNA, 23 nt, Bacillus anthracis
caucaauccauuggaaguaccuu

SEQ ID NO: 11 - DNA, 21 bp, Bacillus anthracis
  misc_feature (20)..(21): desoxynucleotide bases
ucaauccauuggaaguacctt

SEQ ID NO: 12 - DNA, 21 bp, Bacillus anthracis
  misc_feature (20)..(21): desoxynucleotide bases
gguacuuccaauggauugatg